(19) United States
(12) Patent Application Publication
(10) Pub. No.: US 2011/0270239 A1
(43) Pub. Date: Nov. 3, 2011

(54) TRANSSEPTAL CROSSING DEVICE

(76) Inventor: Randell L. WERNETH, San Diego, CA (US)

(21) Appl. No.: 12/769,902

(22) Filed: Apr. 29, 2010

(51) Int. Cl. A61B 18/18; A61B 17/34

(52) U.S. Cl. 606/33; 606/185

(57) ABSTRACT

A medical device for crossing a tissue wall and methods of use thereof are provided. The device may include a flexible shaft defining a proximal portion, a distal portion, and a lumen extending therebetween to apply a vacuum to at least a portion of the tissue wall; and a tissue penetration element movably positioned within the shaft. The device may further include one or more anchors, one or more expandable elements disposed thereon, an image capture device coupled to a portion thereof, an expandable array, and/or one or more electrically conductive segments.

### CROSS-REFERENCE TO RELATED APPLICATION

n/a

### STATEMENT REGARDING FEDERALLY SPONSORED RESEARCH OR DEVELOPMENT

n/a

### FIELD OF THE INVENTION

The present invention relates to systems and methods for intracardiac transseptal access and catheterization.

### BACKGROUND OF THE INVENTION

Transseptal puncture devices are used by physicians who perform specialized invasive cardiology techniques such as angioplasty, tissue ablation, and other diagnostic and therapeutic procedures in the left atrium of a heart. In order to locate the precise area of the septal wall to be punctured, the physician may use fluoroscopic landmarks or ultrasound visualization to identify the various anatomical boundaries that form the septum. For example, localization of the tip of the needle can be detected by the surgeon using various anatomical landmarks around the septum. In particular, the septum is comparatively larger than the fossa ovalis in healthy patients providing an imprecise but important physiological marker. However, in patients with atrial abnormalities, for example a dilated atrium, or as a result of previous surgeries, the traditional markers may change, making localization difficult and increasing the risk of harm to the patient.

Accordingly, angiographic techniques have been devised to ameliorate such drawbacks. For example, transesophageal and transthoracic echocardiography, intravascular ultrasound, and intracardiac echocardiography have all been employed as a means of determining the optimal transseptal puncture site. Transthoracic ultrasound, however, may not be capable of accurately locating the thin wall of the fossa ovalis and presents difficulties in maintaining both patient comfort and sterility, thus often resulting in an increased cost for a given procedure. Transesophageal echocardiography also presents several disadvantages in some cases, such as limited communication with the patient (resulting from sedation), a risk of esophageal bleeding, longer procedure times, additional cost, and inaccurate identification of the fossa ovalis. Lastly, intracardial echocardiography is highly expensive and greatly increases the overall time of the procedure.

Therefore, what is needed is a low-cost, accurate, and safe transseptal crossing device and methods of use thereof that address the drawbacks of current devices and methods.

### SUMMARY OF THE INVENTION

The present invention advantageously provides a medical device for crossing a tissue wall, including a flexible shaft defining a proximal portion, a distal portion, and a lumen extending therebetween to apply a vacuum to at least a portion of the tissue wall; and a tissue penetration element movably positioned within the shaft. The device may include at least one anchor extendable from the distal portion to engage the tissue wall; a hood coupled to the distal portion of the shaft; a cavity at the distal portion in fluid communication with the lumen; an expandable array extendable from the distal portion of the shaft; a first expandable element coupled to the distal portion of the shaft; a second expandable element coupled to the distal portion of the shaft, where the first expandable element is inflatable independently of the inflation of the second expandable element, and where at least one of the first and second expandable elements defines a tapered profile. The device may also include an electrically conductive segment disposed on the shaft; a tensioning element such as a spring coupled to the penetration element that controllably extends and retracts the penetration element with respect to the shaft; an image capture device coupled to the distal portion of the shaft; a guide movably disposed within the shaft, the guide being extendable from the shaft to abut a proximal portion of the penetration element. Further, the shaft may define a collar and the penetration element may define a flange, such that the collar limits a range of movement of the penetration element by contacting the flange.

A method for piercing a transseptal wall is also provided, including positioning a flexible shaft proximate the transseptal wall, the shaft defining a distal portion and a lumen therein; and ejecting a fluid from the distal portion to pierce the transseptal wall. The method may also include evacuating at least some of the fluid through the shaft.

A method of treating cardiac tissue is also provided, including positioning a tissue crossing device proximate a septal wall, the tissue crossing device having a penetration element movably disposed therein, the tissue crossing device further including an electrically conductive segment; creating an opening in the septal wall with the penetration element; maneuvering a radiofrequency treatment device through the hole; and emitting a radiofrequency signal from the treatment device to treat cardiac tissue, wherein at least a portion of the emitted signal is conducted through the electrically conductive segment of the tissue crossing device. The method may include engaging at least a portion of the septal wall with the tissue crossing device to prior to creation of the opening, where the engagement includes the application of a vacuum through at least a portion of the tissue crossing device.

### BRIEF DESCRIPTION OF THE DRAWINGS

A more complete understanding of the present invention, and the attendant advantages and features thereof, will be more readily understood by reference to the following detailed description when considered in conjunction with the accompanying drawings wherein:

FIG. 1a is a cross-sectional illustration of an exemplary transseptal penetration device constructed in accordance with the principles of the present invention;

FIG. 1b is a distal end view of the transseptal penetration device of FIG. 1a;

FIG. 1c is an illustration of the transseptal penetration device of FIG. 1a in proximity to a tissue wall;

FIG. 1d is another illustration of the transseptal penetration device of FIG. 1a in proximity to a tissue wall;

FIG. 2 is an additional illustration of the transseptal penetration device of FIG. 1a with a distal cover;

FIG. 3a is a cross-sectional illustration of another exemplary transseptal penetration device constructed in accordance with the principles of the present invention;

FIG. 3b is a distal end view of the transseptal penetration device of FIG. 3a;

FIG. 4a is a cross-sectional illustration of another exemplary transseptal penetration device constructed in accordance with the principles of the present invention;

FIG. 4b is a distal end view of the transseptal penetration device of FIG. 4a;

FIG. 4c is an illustration of the transseptal penetration device of FIG. 4a in proximity to a tissue wall;

FIG. 4d is another illustration of the transseptal penetration device of FIG. 4a in proximity to a tissue wall;

FIG. 5a is a cross-sectional illustration of an exemplary transseptal penetration device having one or more anchors constructed in accordance with the principles of the present invention;

FIG. 5b is a distal end view of the transseptal penetration device of FIG. 5a;

FIG. 5c is an illustration of the transseptal penetration device of FIG. 5a in proximity to a tissue wall;

FIG. 5d is another illustration of the transseptal penetration device of FIG. 5a in proximity to a tissue wall;

FIG. 6 is a cross-sectional illustration of another exemplary transseptal penetration device having one or more anchors constructed in accordance with the principles of the present invention;

FIG. 7 is a cross-sectional illustration of still another exemplary transseptal penetration device having one or more anchors constructed in accordance with the principles of the present invention;

FIG. 8 is an illustration of another exemplary transseptal penetration device having one or more anchors constructed in accordance with the principles of the present invention;

FIG. 8a is a distal end view of the transseptal penetration device of FIG. 8;

FIG. 9 is an illustration of another exemplary transseptal penetration device with a distal array constructed in accordance with the principles of the present invention;

FIG. 10a is a cross-sectional illustration of yet another exemplary transseptal penetration device constructed in accordance with the principles of the present invention;

FIG. 10b is an additional illustration of the transseptal penetration device of FIG. 10a;

FIG. 10c is another illustration of the transseptal penetration device of FIG. 10a in proximity to a tissue wall;

FIG. 10d is still another illustration of the transseptal penetration device of FIG. 10a in proximity to a tissue wall;

FIG. 11a is a cross-sectional illustration of yet another exemplary transseptal penetration device constructed in accordance with the principles of the present invention;

FIG. 11b is an additional illustration of the transseptal penetration device of FIG. 11a;

FIG. 11c is another illustration of the transseptal penetration device of FIG. 11a in proximity to a tissue wall;

FIG. 12a is a cross-sectional illustration of yet another exemplary transseptal penetration device constructed in accordance with the principles of the present invention;

FIG. 12b is an additional illustration of the transseptal penetration device of FIG. 12a;

FIG. 12c is another illustration of the transseptal penetration device of FIG. 12a in proximity to a tissue wall;

FIG. 13 is an illustration of an exemplary transseptal penetration device having an inflatable element constructed in accordance with the principles of the present invention;

FIG. 14 is an illustration of still another exemplary transseptal penetration device having one or more inflatable elements constructed in accordance with the principles of the present invention;

FIG. 15a is a cross-sectional illustration of an exemplary transseptal penetration device implementing discharged fluid constructed in accordance with the principles of the present invention;

FIG. 15b is an additional illustration of the transseptal penetration device of FIG. 15a;

FIG. 15c is another illustration of the transseptal penetration device of FIG. 15a in proximity to a tissue wall;

FIG. 16a is a cross-sectional illustration of an exemplary transseptal penetration device constructed in accordance with the principles of the present invention;

FIG. 16b is an additional illustration of the transseptal penetration device of FIG. 16a;

FIG. 16c is another illustration of the transseptal penetration device of FIG. 16a in proximity to a tissue wall;

FIG. 17a is a cross-sectional illustration of yet another exemplary transseptal penetration device having an image capturing device constructed in accordance with the principles of the present invention; and

FIG. 17b is an additional illustration of the transseptal penetration device of FIG. 17a;

### DETAILED DESCRIPTION OF THE INVENTION

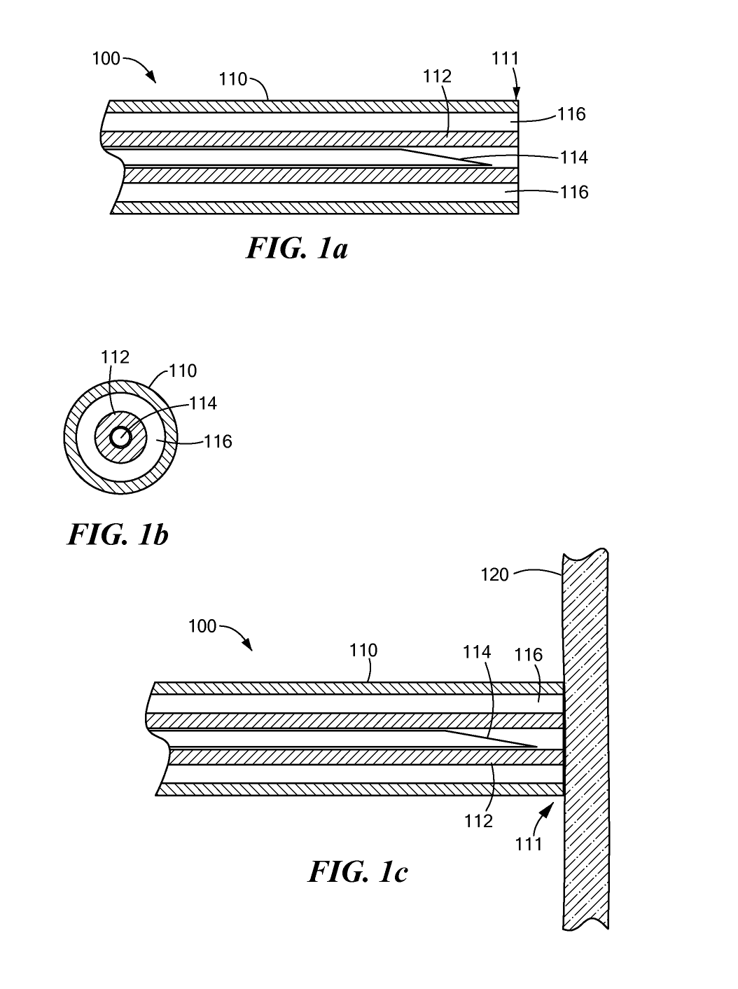

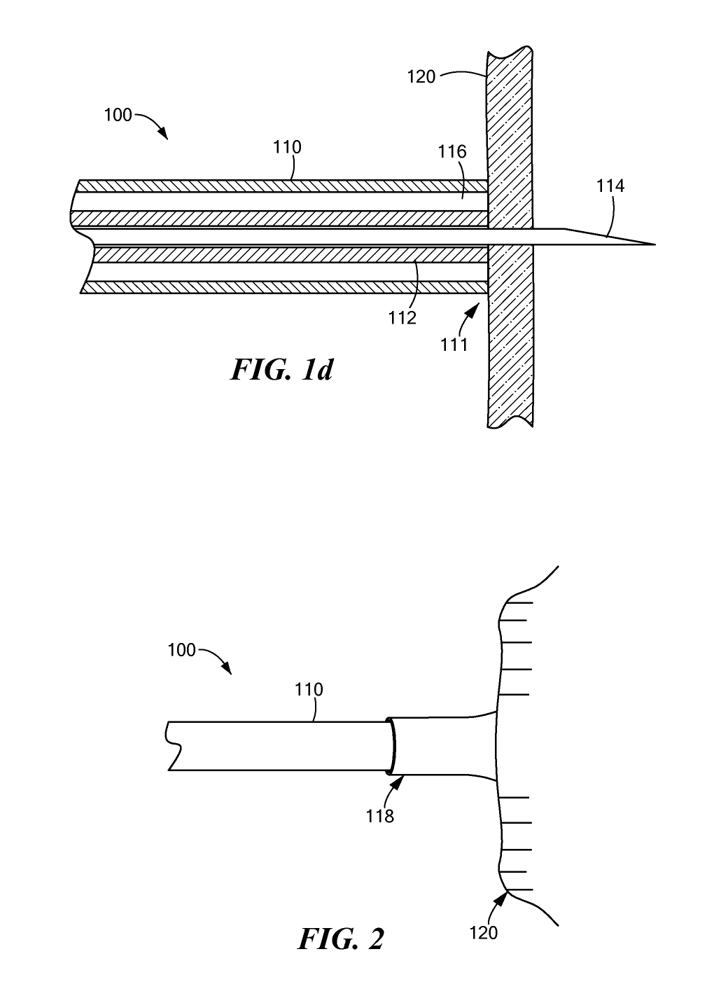

The present invention provides transseptal penetration and crossing devices and methods of use thereof. Referring now to the drawings in which like reference designators refer to like elements, there is shown in FIGS. 1a-1d an exemplary embodiment of a tissue crossing or penetration device constructed in accordance with the principles of the present invention, designated generally as 100. Of note, the device components have been represented where appropriate by conventional symbols in the drawings, showing only those specific details that are pertinent to understanding the embodiments of the present invention so as not to obscure the disclosure with details that will be readily apparent to those of ordinary skill in the art having the benefit of the description herein. Moreover, while certain embodiments or figures described herein may illustrate features not expressly indicated on other figures or embodiments, it is understood that the features and components of the penetration device disclosed herein may be included in a variety of different combinations or configurations without departing from the scope and spirit of the invention.

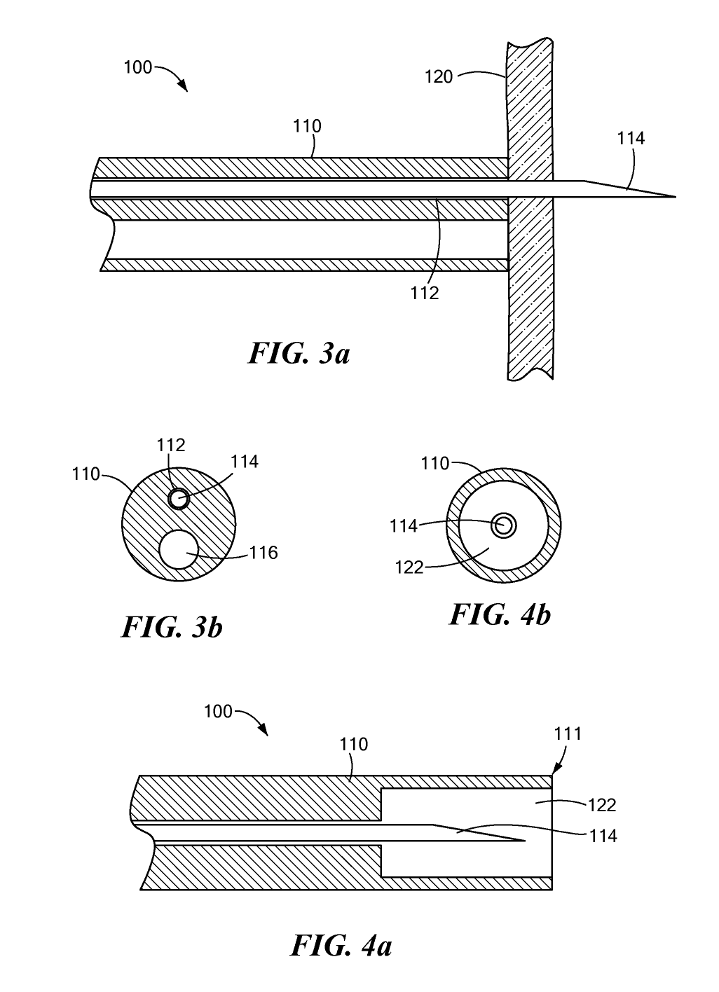

Continuing to refer to FIGS. 1a-1d, the penetration device 100 may generally include an elongate body or shaft 110 that is insertable or otherwise positionable through the vasculature of a patient. The shaft 110 may include one or more lumens disposed or otherwise defined therein for fluid circulation, vacuum application, steering elements, or other components and characteristics for use. For example, the shaft 110 may define a proximal portion (not shown) and a distal portion 111. An interior shaft, body, or other conduit may be disposed within the shaft 110 having a first lumen 112 defined therein extending through at least a portion of the shaft 110. The first lumen 112 may be centralized within the shaft 110 parallel to a longitudinal axis of the shaft 110 (as shown in FIGS. 1a-1d), or may be arrange in an eccentric configuration with respect to the shaft 110 and/or other lumens therein (as shown in FIGS. 3a-3b).

A penetration element 114 may be at least partially disposed within the first lumen 112 at the distal portion 111 of the device 100 to facilitate or otherwise achieve the puncturing or piercing of a targeted tissue structure, such as the fossa ovalis for example. The penetration element 114 may include a sharpened, edged, or otherwise formed construct able to cut through tissue, including a blade, needle or the like.

The penetration device 100 may further include a second lumen 116 within the shaft 110 that is coaxially aligned with the first lumen 112. The second lumen 116 may be defined by an additional conduit disposed within the shaft 110, or may be defined by the shaft 110 itself. The second lumen 116 may be in fluid communication with a vacuum source to provide for a controlled or desired amount of suction at the distal region of the device 100 in order to securely engage a targeted tissue structure. To further facilitate engagement of a selected tissue region, the device 100 may include a cover or hood 118 extending from the distal portion 111 of the shaft 110, as shown in FIG. 2. The hood 118 may define an increased diameter to that of the distal portion 111 of the shaft 110 to engage a larger circumference or area of tissue. The distal portion 111 of the shaft 110 may then be maneuvered or steered within the engaged hood 118 to a location where piercing the tissue is desired.

For example, as shown in FIG. 1c, the device 100 may be positioned directly adjacent a tissue wall 120, such as the fossa ovalis extending between the right and left atrium, aided by the application of a vacuum to the tissue via the second lumen 116 and/or the hood. Once in the desired position, the penetration element 114 may be extended from the device 100 through the tissue wall 120, as shown in FIG. 1d. The application of a vacuum or suction to the targeted tissue by the device 100 aids in both tensioning the tissue to allow easier penetration as well as preventing the pierced tissue from “tenting” or extending outward from the opposite side of the tissue wall 120 (into the left atrium, for example), thereby decreasing the likelihood of any complications or unintended trauma to surrounding tissue structures from the tissue penetration. Once the tissue has been crossed or otherwise pierced, the vacuum/suction application may be discontinued.

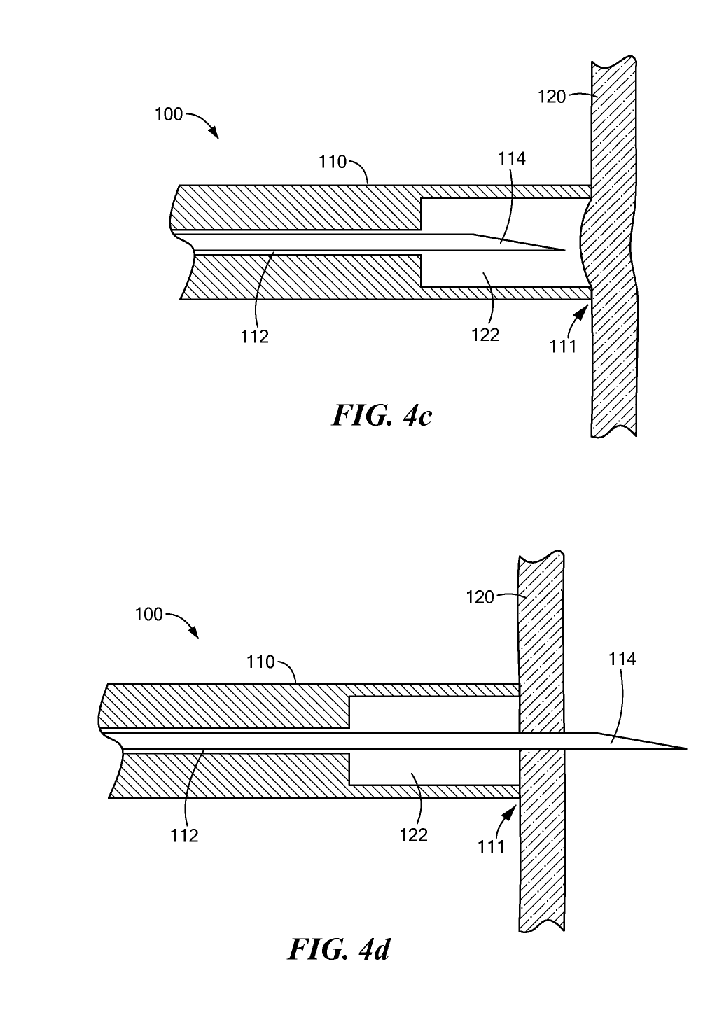

FIGS. 4a and 4b illustrate an additional configuration of the penetration device 100. In particular, the distal portion 111 may include a cavity 122 in fluid communication with a vacuum source via the first and/or second lumen 116s. For example, the first lumen 112 illustrated in FIGS. 4a and 4b may be in fluid communication with a vacuum source and also include a portion of the penetrating device 100 therein. The cavity 122 may provide a larger circumference for engaging targeted tissue than that that of the first or second lumens to facilitate secure engagement of the targeted tissue site. As shown in FIGS. 4c and 4d, the distal portion 111 of the device 100 may again be positioned in proximity to the targeted tissue wall 120, where the application of a vacuum or suction may draw the targeted tissue into the cavity 122 of the distal portion 111 of the device 100. The penetration element 114 may subsequently be extended from the distal portion 111 of the device 100 and through the entrained tissue section to provide a passage therethrough for subsequent instruments or therapeutic elements. Of note, such extension of the penetration element 114 may be achieved during application of a vacuum through the first lumen 112 by providing sufficient force to the penetration element 114 to overcome the applied vacuum force.

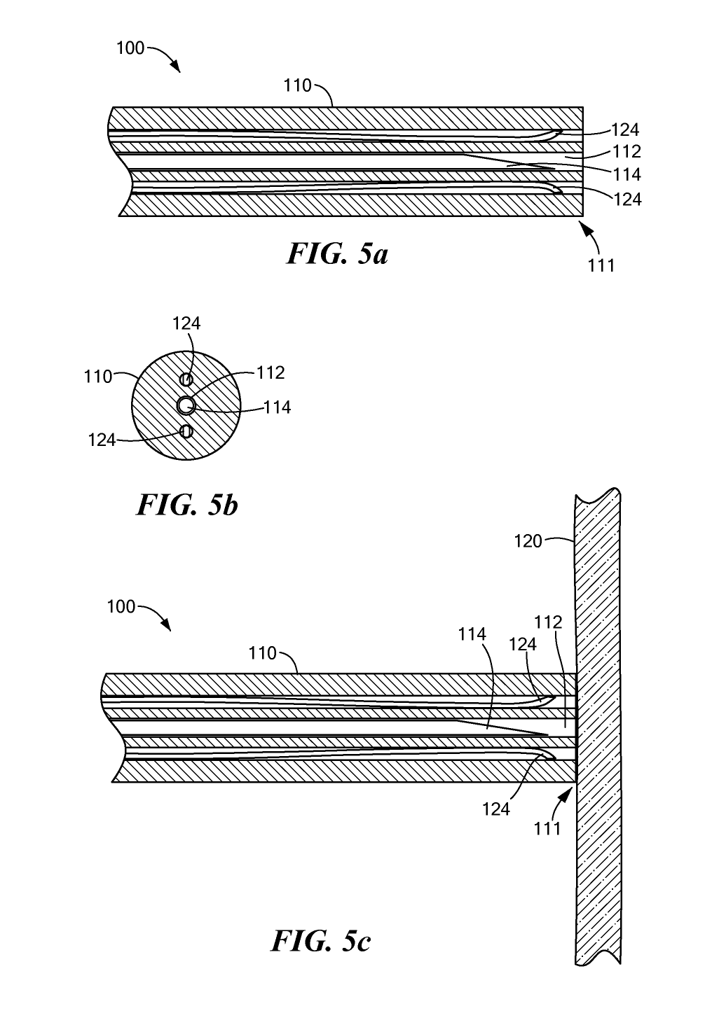

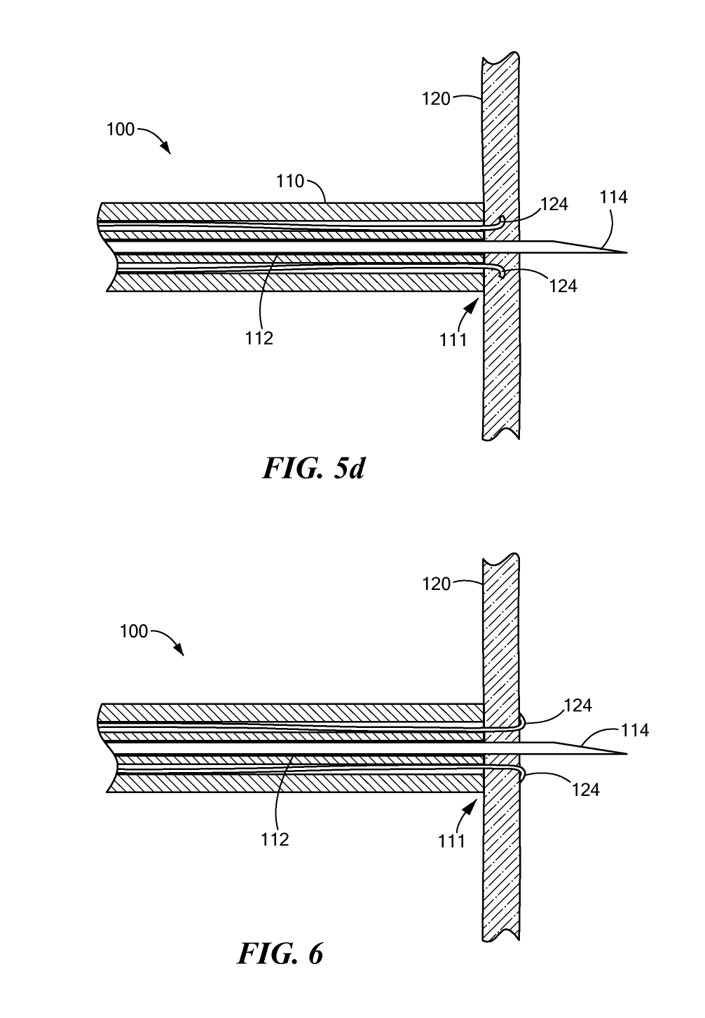

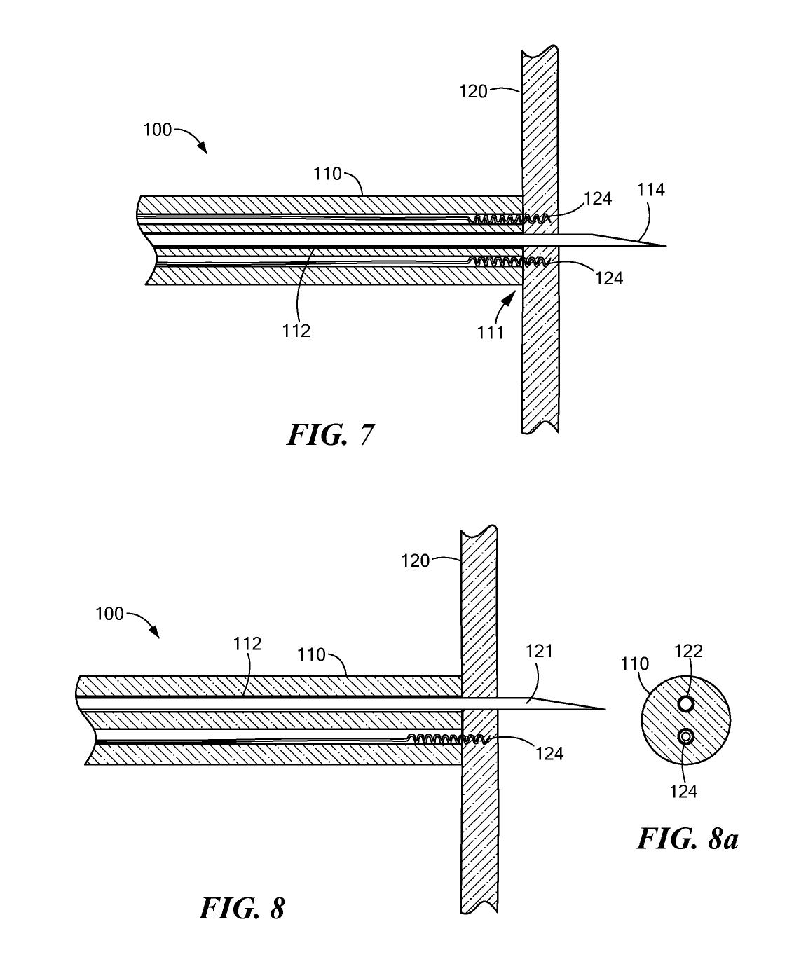

The penetration device 100 may further include one or more anchors 124 for releasably attaching the distal portion 111 of the shaft 110 to a targeted tissue wall 120 or region. For example, as shown in FIGS. 5a-5b, the device 100 may include one or more anchors 124 at least partially disposed within additional lumens defined in the shaft 110. The anchors 124 may include strands of filament or other structures having sufficient rigidity to be at least partially embedded into a tissue region. The anchors 124 may be controllably distended and retracted from the distal portion 111 of the shaft 110 by one or more controls at the proximal portion of the device 100. Now referring to FIGS. 5c-5d, the distal portion 111 may be positioned in proximity to the tissue wall 120, and the anchor(s) 124 may be longitudinally moved outward from the distal portion 111 of the shaft 110 to at least partially engage the tissue structure, thereby securing the device 100 to the targeted tissue structure for subsequent penetration and/or crossing by the penetration element 114, as shown in FIG. 5d. Additional variations in the characteristics of the anchor(s) 124 and/or its placement within the device 100 are also contemplated. For example, FIGS. 6 and 7 illustrate the device 100 having anchors 124 extending through the tissue wall 120 and including a helical or other screw-like configuration, respectively, to aid in attaching the device 100 to the tissue. FIGS. 8 and 8a illustrate the shaft 110 of the device 100 including the first lumen 112 with the penetration element 114 being offset or otherwise eccentric with respect to the lumen containing an anchor therein. Irrespective of the particular configuration, once the tissue wall 120 is pierced (and/or any subsequent treatment or procedure is completed), the anchor(s) 124 may be controllably retracted into the shaft 110 for removal and/or repositioning of the device 100.

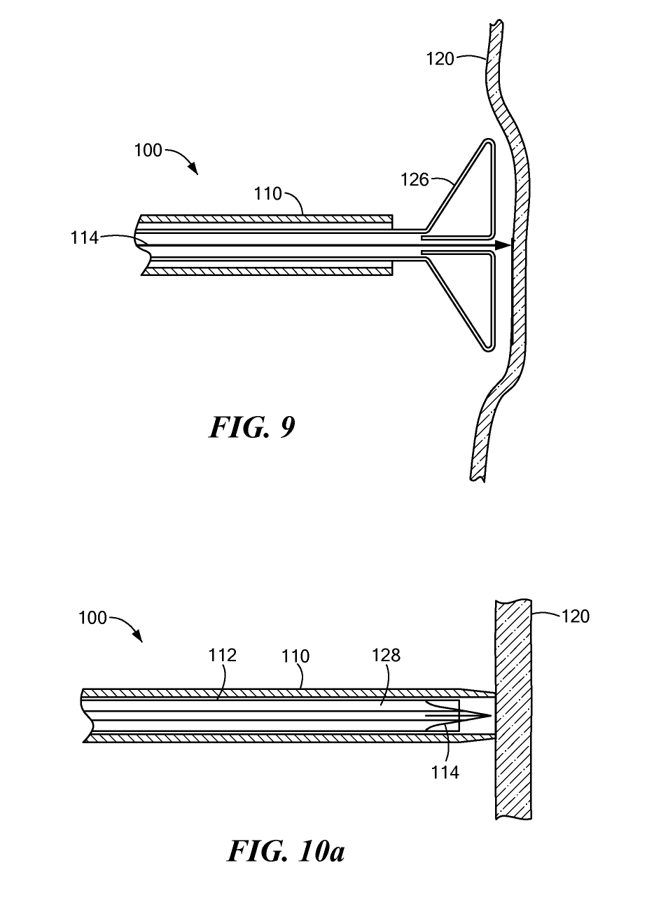

Now turning to FIG. 9, the penetration device 100 may include a distal array or assembly 126 extendable from the distal portion 111 of the shaft 110 that can aid in locating an approximate center or target region of a portion of a tissue wall 120. For example, the distal array 126 may include a plurality of expandable arms that can engage or otherwise abut a portion of a tissue region. In particular, the fossa ovalis is a depression in the septum between the right and left atria. The array 126 may be maneuvered about the fossa ovalis until it is positioned within the depression of the tissue, e.g., by fitting the center of the array 126 into the center of the fossa ovalis. If the array 126 were not within the boundaries of the fossa ovalis, the edges of the array 126 would be out of plane from the portions that are within the fossa ovalis area. The difference in planar positioning, thus indicating the proximity/positioning of the array 126 to the fossa ovalis, may be provided visually through fluoroscopic or other imaging means, or may be provided through tactile feedback or positioning of one or more components of the device 100 at its proximal end.

Once the desired location for crossing the tissue wall 120 has been identified, the penetration element 114 may be extended towards and through the tissue wall 120, as described above.

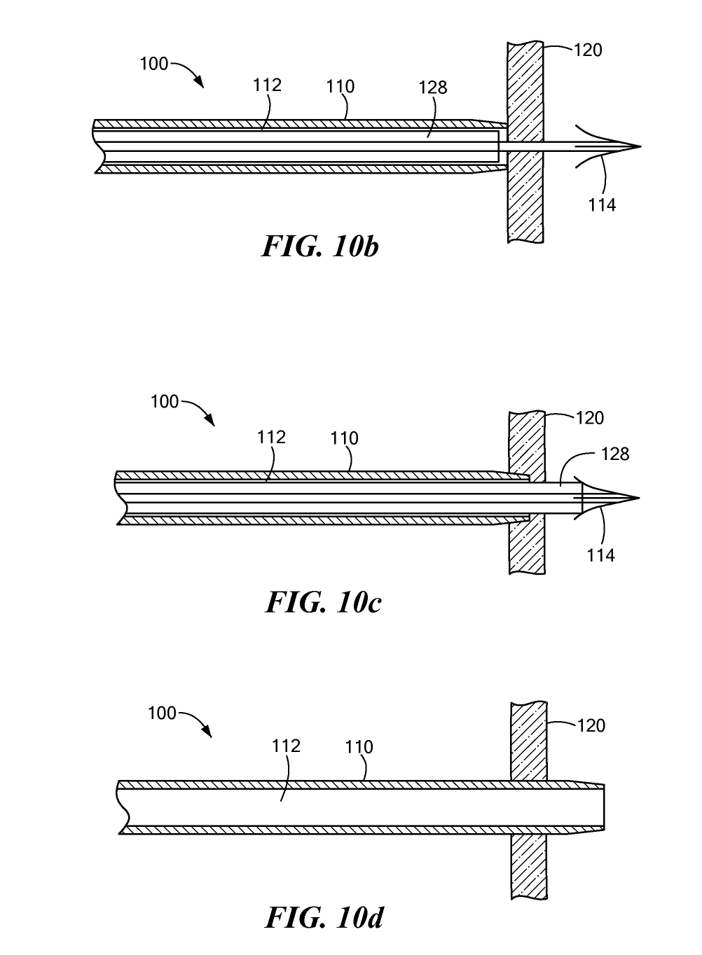

Now referring to FIGS. 10a-10d, the penetration device 100 may be configured to limit or otherwise control passage of a portion of the device 100 through an accessed tissue wall 120 to prevent overextension or positioning into a region opposite the punctured wall, which could result in injury. For example, the penetration element 114 illustrated in FIGS. 10a-10d includes an arrow-like shape defining a distal tip and a proximal region. The proximal region of the penetration element 114 may be used to limit or stop travel of additional portions of the device 100 into an adjacent cavity opposite the traversed tissue wall 120. In particular, the device 100 may include an extendable sheath or guide 128 movably positioned within at least a portion of the shaft 110. The guide 128 may be coaxially disposed about the penetration element 114 and movable with respect thereto. In an exemplary use, the penetration element 114 is again pierced through the tissue wall 120 and into an adjacent cavity or region, as shown in FIG. 10b. Once the penetration element 114 has been extended through the tissue wall 120, the guide 128 may be moved along the penetration element 114 through the tissue wall 120 until it abuts the proximal portion of the penetration element 114, thus preventing the guide 128 from moving past the penetration element 114 and potentially damaging surrounding structures, as shown in FIG. 10c. The shaft 110 of the device 100 may subsequently be moved through the tissue wall 120 to a position adjacent the guide 128 to ensure that the shaft 110, too, does not undesirably move further into the adjacent cavity than is needed. The depth and/or positioning of the penetration element 114, guide, and/or shaft 110 may be initially aided by fluoroscopy or other imaging techniques to place any of these components through the tissue wall 120, with subsequent placement or positioning being limited by the proximal portion of the penetration element 114. The guide 128 and/or the penetration element 114 may subsequently be removed, as shown in FIG. 10d, to allow one or more therapeutic or diagnostic device 100s to pass through the shaft 110.

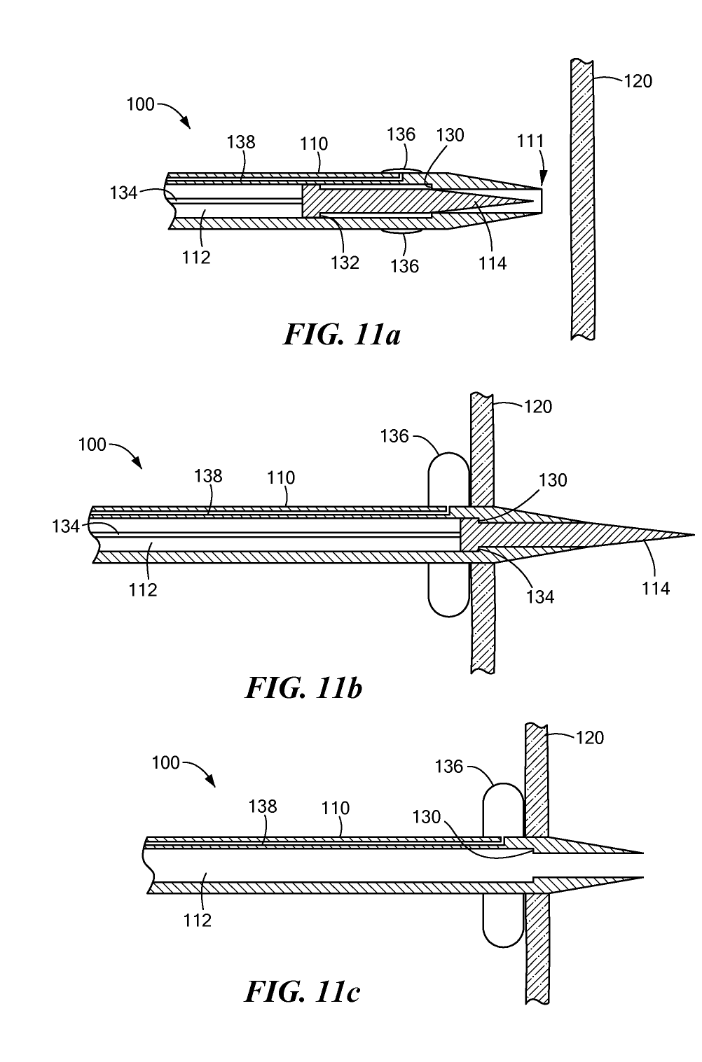

Now referring to FIGS. 11a-11c, the penetration device 100 may include a distal portion 111 that is tapered or otherwise defines a smaller circumferential profile than a more-proximal portion of the device 100 to facilitate transitioning through a targeted, punctured tissue wall 120. The tapered portion may extend towards an endpoint at which the taper of the distal portion 111 matches a taper or angled edge of the penetration element 114 when the penetration element 114 is extended, as shown in FIG. 11b. The substantial similarity and relative seamlessness between the respective tapers of the distal portion 111 of the shaft 110 and the penetration element 114 aids in easing passage of the device 100 through a tissue wall 120.

The penetration device 100 shown in FIGS. 11a-11c may further limit the longitudinal extension of the penetration element 114 to ensure the penetration element 114 does not undesirably extend past a certain distance from the distal portion 111 of the device 100. For example, the penetration device 100 may include a collar or shelf 130 in its distal portion 111 that abuts a complimentary feature, such as an enlarged circumferential flange 132, of the penetration element 114 when the penetration element 114 is extended to its maximum desired distance from the shaft 110. During use, the penetration element 114 may be extended and retracted out of and into the distal portion 111 of the shaft 110 through by manipulating a control arm 134 coupled to the penetration element 114. The control arm 134 may be coupled to one or more controls at the proximal portion of the device 100, such as a knob, lever, sliding button, or the like. When extending the penetration element 114 distally out of the shaft 110, at least a portion of the penetration element 114 abuts the shelf to inhibit any further extension.

To secure the positioning of the device 100 and/or to prevent the device 100 from moving when the penetration element 114 abuts the shelf, the penetration device 100 may include a stabilizing element able to engage the targeted tissue wall 120 or to otherwise prevent unwanted movement of the device 100 with respect to the tissue wall 120 during use. For example, the device 100 may include an expandable or inflatable element 136, such as a balloon, coupled to a distal portion 111 of the shaft 110. The device 100 may further include an inflation lumen 138 disposed in or about the shaft 110 for the controllable inflation or introduction of fluid into the expandable element. In use, the device 100 may be positioned proximate a desired region of a tissue wall 120 for crossing or piercing. Once positioned, a fluid may be introduced into the expandable element 136 to expand it, such that the expandable element abuts a larger region of the tissue wall 120 than that of the shaft 110. Positioning of the expandable element 136 adjacent a segment of the tissue wall 120 being breached reduces the likelihood that the device 100 itself will be unintentionally pushed through the tissue wall 120 too far into the adjacent region, which could cause injury to surrounding tissue structures. Moreover, because the shaft 110 of the device 100 is secured in place by the expandable element 136, the shaft 110 has increased resistance to inadvertent movement of the device 100 upon actuation or extension of the penetration element 114 and contact between the penetration element 114 and the shelf 130. Once the tissue wall 120 has been breached, the penetration element 114 may be withdrawn (as shown in FIG. 11c) to allow the passage of additional therapeutic or diagnostic device 100s and instruments through the shaft 110.

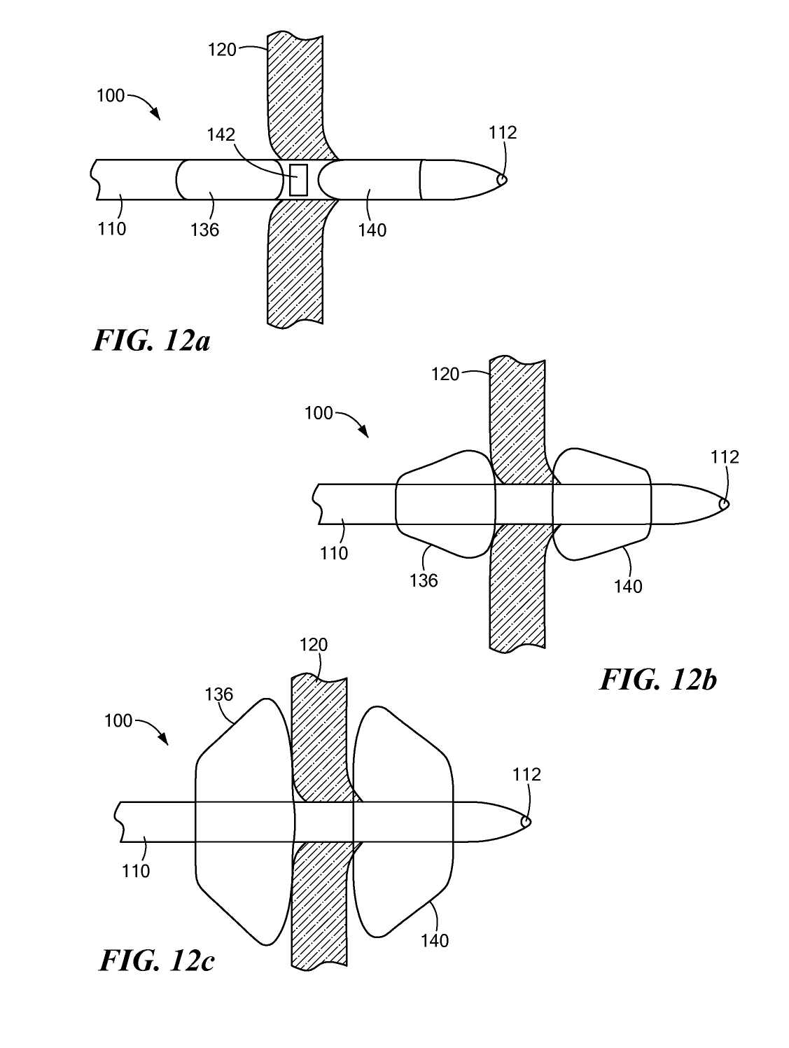

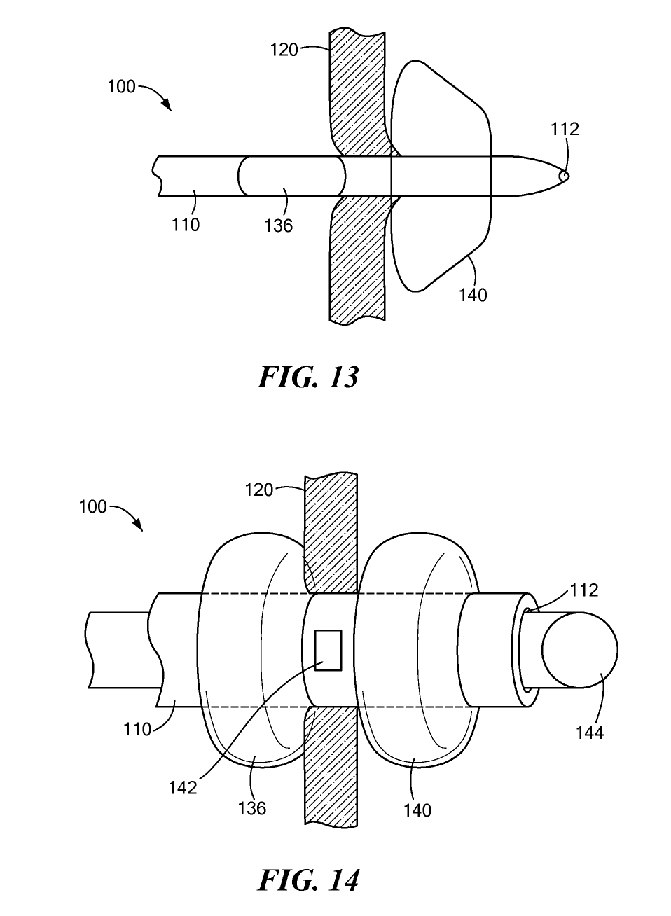

Now turning to FIGS. 12a-12c, the penetration device 100 is shown having a plurality of expandable elements on its distal portion 111 to aid in securing the positioning of the device 100. In particular, the device 100 may include a first expandable element 136, such as a balloon, coupled to the distal portion 111 of the device 100 that is positionable adjacent to a proximal side of the tissue wall 120. The penetration device 100 may further include a second expandable element 140 positionable adjacent to a distal side of the tissue wall 120. Either of the first and second expandable elements may define a tapered construction such that a larger radius or circumference of the balloon abuts the tissue wall 120 while an end of the expandable elements located away from the tissue wall 120 defines a smaller radius or circumference. The tapered profiles decrease the amount of inflation medium required for inflation while maintaining an increased contact surface with the tissue wall 120 to secure the device 100 in place. The first and second expandable elements may be in fluid communication with first and second inflation lumens providing for the controlled delivery and evacuation of a fluid from the first and second expandable elements either simultaneously or independently. By expanding or otherwise positioning the first and second expandable elements 136, 140 about opposing sides of the traversed tissue wall 120 (i.e., “sandwiching” the traversed tissue), the likelihood that the device 100 will be unintentionally moved, dislodge, or otherwise displaced from its desired, initial position during subsequent use or introduction of device 100s through the shaft 110 is reduced. Moreover, independent inflation of the first and second expandable elements allows either expandable element to be inflated selectively depending on the stage of treatment or positioning of additional device 100s about the penetration device 100. For example, when removing devices from within the shaft 110, it may be desirable to only inflate or maintain inflation of the second expandable element 140 (as shown in FIG. 13) to resist pull back of the penetration device 100 while removing one or more device 100s from the shaft 110.

Again referring to FIG. 12a, the distal portion 111 of the penetration device 100 may include an electrically conductive segment 142 on a portion of the shaft 110 for use in a subsequent energy-based treatment. For example, the electrically conductive segment 142 may include an electrode or other electrically-conductive deposit on a portion of the shaft 110. As discussed above, upon crossing the tissue wall 120, additional devices may be introduced through the shaft 110 for treatment and/or diagnosis of a given condition. Such devices may include those for delivering radiofrequency energy (“RF”) to the tissue, for example. RF devices sometimes employ a “ground” electrode or “return” electrode to complete a circuit and path for energy to travel in order to effect the desired treatment. Now turning to FIG. 14, the electrically conductive segment 142 of the shaft 110 of the penetration device 100 can serve as the return electrode during operation of an RF device 144 disposed within or otherwise traversing a portion of the shaft 110 of the penetration device 100. Alternatively, the electrically conductive segment 142 may be used as an active electrode that emits an RF signal to the surrounding tissue and/or another device 100 or component located on or about the patient. To facilitate operation or use during an RF procedure, the electrically conductive segment 142 on the shaft 110 may be electrically coupled to one or more wires on an interior portion of the shaft 110 that communicate signals to and from one or more instruments or control device 100s at a proximal portion of the device 100.

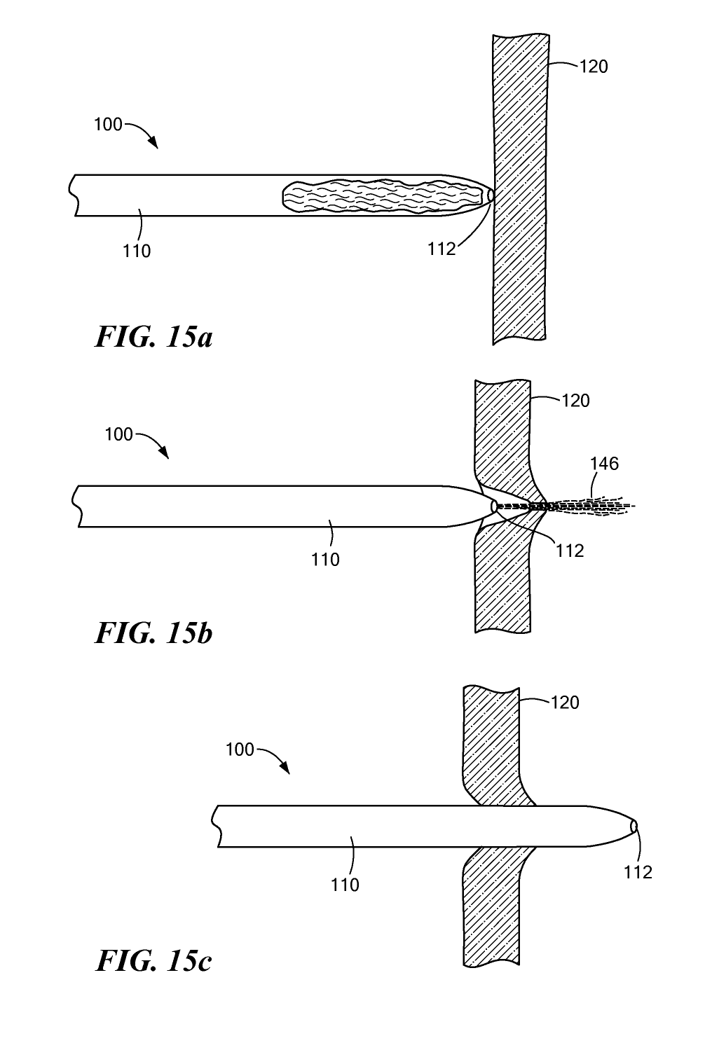

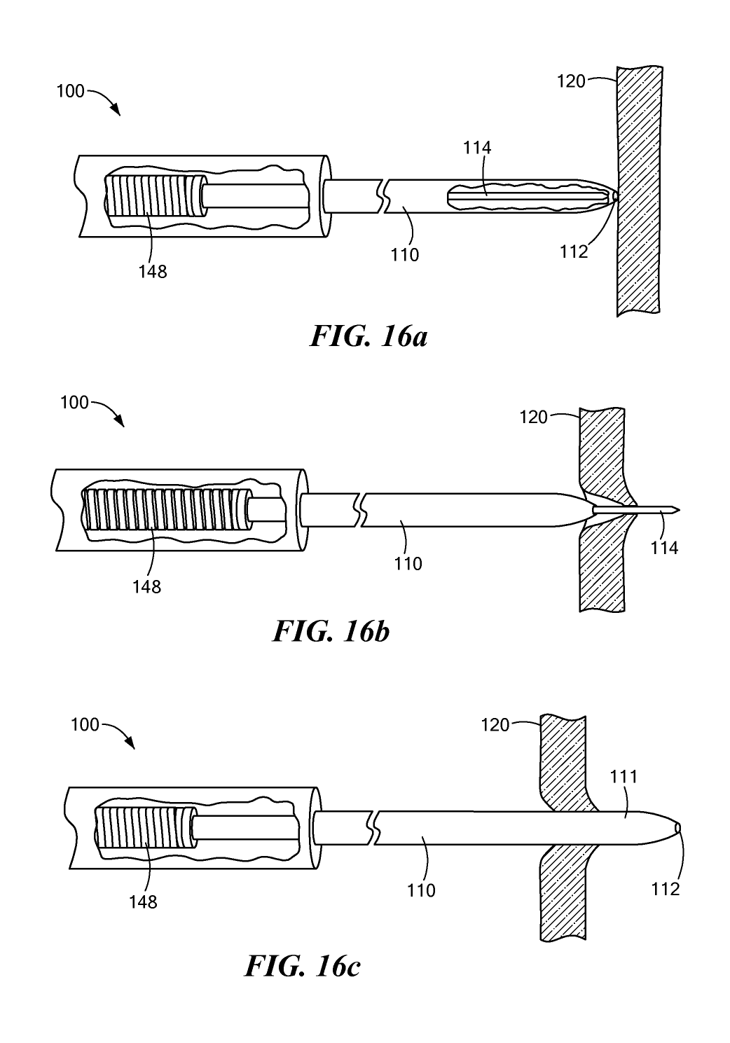

Now referring to FIGS. 15a-15c, the distal portion 111 of the penetration device 100 includes a tapered geometry to aid in passing through a portion of a targeted tissue wall 120. This exemplary embodiment includes the high-pressure delivery of a fluid 146 through the shaft 110 to pierce or otherwise traverse the target tissue wall 120. For example, the shaft 110 or a lumen therein may be placed into fluid communication with a pressurized fluid source (not shown) at the proximal portion of the device 100. The pressurized fluid 146 may be introduced or otherwise circulated through the shaft 110 to the distal portion 111 of the device 100, where the fluid may be dispersed or streamed out of the device 100 at sufficient pressure and force to penetrate the tissue wall 120, thereby allowing the penetration device 100 to pass through an opening created therein. The fluid may include water, saline, or other suitable non-toxic, biocompatible compositions. The delivery of fluid to the distal portion 111 of the device 100 may include the operation of one or more valves, pumps, or other fluid-control components within the device 100 or coupled to a proximal portion thereof, as known in the art. Moreover, the shaft may define one or more lumens therein that are coupled to a vacuum source or otherwise able to evacuate or aspirate fluids away from the region where the crossing or tissue piercing was affected.

Turning now to FIGS. 16a-16c, the penetration device 100 is shown having an actuation mechanism at its proximal portion for controllably extending and retracting the penetration element 114 at the distal portion 111 of the device 100. The actuation mechanism may include a tensioning element 148 such as a spring, elastic bands, or other mechanical-energy-storing component that can be used to power or drive the extension of the penetration element 114 from the distal portion 111 of the penetration device 100. For example, as shown in FIG. 16a, the tensioning element 148 is in a retracted or untriggered state, and the penetration element 114 is remains within the shaft 110 of the device 100. In FIG. 16b, the tensioning element 148 has been released or otherwise triggered to forcefully cause the penetration element 114 to extend out form the distal portion 111 of the device 100 and through the targeted tissue wall 120. The penetration element 114 may subsequently be drawn back into the shaft 110 and the tensioning element 148 may be reset to its initial retracted or untriggered state. The operation or manipulation of the tensioning element 148 and thus the penetration element 114 may be facilitated by one or more controls (not shown) at the proximal portion of the device 100 that are coupled to the tensioning element and/or the penetration element 114.

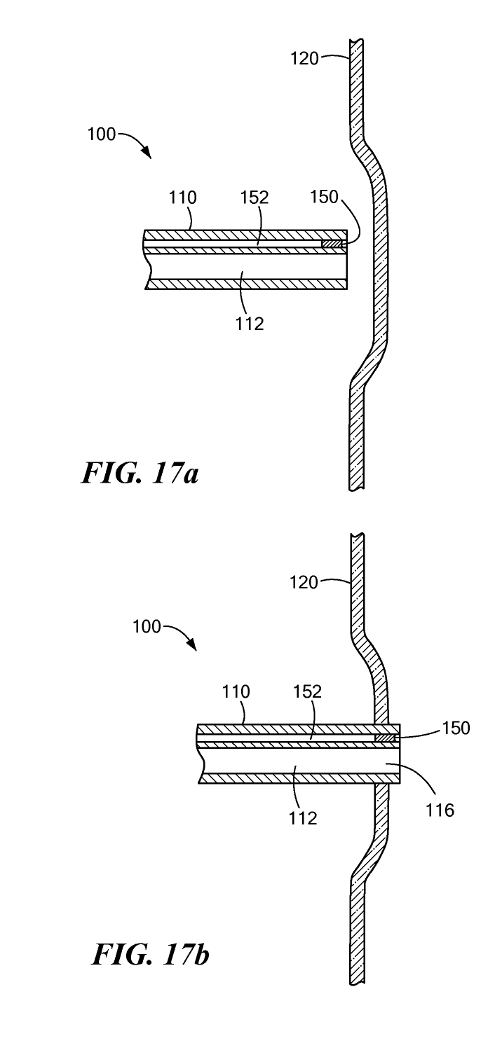

Referring now to FIGS. 17a and 17b, the penetration device 100 may include an image capture device 150 coupled to its distal portion 111 to visually monitor or locate a desired position for crossing a targeted tissue structure, such as the fossa ovalis for example. The image capture device 150 may include a camera in communication with a processor, monitor or the like at the proximal portion of the device 100 to provide captured images to an operating physician or operator. The communication may be provided, for example, through a conduit or cable 152 transmitting an electrical, optical, or other communicative signal from the distal portion 111 of the device 100 to the proximal portion.

It will be appreciated by persons skilled in the art that the present invention is not limited to what has been particularly shown and described herein above. In addition, unless mention was made above to the contrary, it should be noted that all of the accompanying drawings are not to scale. A variety of modifications and variations are possible in light of the above teachings without departing from the scope and spirit of the invention, which is limited only by the following claims.

What is claimed is:

1. A medical device for crossing a tissue wall, comprising:
a flexible shaft defining a proximal portion, a distal portion, and a lumen extending therebetween to apply a vacuum to at least a portion of the tissue wall; and
a tissue penetration element movably positioned within the shaft.

2. The device according to claim 1, further comprising at least one anchor extendable from the distal portion to engage the tissue wall.

3. The device according to claim 1, further comprising a hood coupled to the distal portion of the shaft.

4. The device according to claim 1, wherein the distal portion of the shaft defines a cavity in fluid communication with the lumen.

5. The device according to claim 1, further comprising an expandable array extendable from the distal portion of the shaft.

6. The device according to claim 1, further comprising a first expandable element coupled to the distal portion of the shaft.

7. The device according to claim 6, further comprising a second expandable element coupled to the distal portion of the shaft.

8. The device according to claim 7, wherein the first expandable element is inflatable independently of the inflation of the second expandable element.

9. The device according to claim 7, wherein at least one of the first and second expandable elements defines a tapered profile.

10. The device according to claim 1, further comprising an electrically conductive segment disposed on the shaft.

11. The device according to claim 1, further comprising a tensioning element coupled to the penetration element that controllably extends and retracts the penetration element with respect to the shaft.

12. The device according to claim 11, wherein the tensioning element is a spring.

13. The device according to claim 1, further comprising an image capture device coupled to the distal portion of the shaft.

14. The device according to claim 1, further comprising a guide movably disposed within the shaft, the guide being extendable from the shaft to abut a proximal portion of the penetration element.

15. The device according to claim 1, wherein the shaft defines a collar and the penetration element defines a flange, and wherein the collar limits a range of movement of the penetration element by contacting the flange.

16. A method for piercing a transseptal wall, comprising:
positioning a flexible shaft proximate the transseptal wall, the shaft defining a distal portion and a lumen therein; and
ejecting a fluid from the distal portion to pierce the transseptal wall.

17. The method according to claim 16, further comprising evacuating at least some of the fluid through the shaft.

18. A method of treating cardiac tissue, comprising:
positioning a tissue crossing device proximate a septal wall, the tissue crossing device having a penetration element movably disposed therein, the tissue crossing device further including an electrically conductive segment;
creating an opening in the septal wall with the penetration element;
maneuvering a radiofrequency treatment device through the hole; and
emitting a radiofrequency signal from the treatment device, wherein at least a portion of the emitted signal is conducted through the electrically conductive segment of the tissue crossing device.

19. The method according to claim 18, further comprising engaging at least a portion of the septal wall with the tissue crossing device to prior to creation of the opening.

20. The method according to claim 19, wherein the engagement includes the application of a vacuum through at least a portion of the tissue crossing device.
